(19) United States
(12) Patent Application Publication
(10) Pub. No.: US 2023/0395208 A1
(43) Pub. Date: Dec. 7, 2023

(54) FEDERATED DATA PLATFORM INTEGRATING MULTIPLE HEALTHCARE DATA SOURCES INCLUDING FHIR AND NON-FHIR SOURCES

(71) Applicant: COMMURE, INC., PALO ALTO, CA (US)

(72) Inventors: Radha Krishanna Mohan KONYALA, Saratoga, CA (US); Edgar Giovanny Gutiérrez Gaitan, Bogota (CO); Abhijit MITRA, Saratoga, CA (US)

(73) Assignee: COMMURE, INC., PALO ALTO, CA (US)

(21) Appl. No.: 17/805,523

(22) Filed: Jun. 6, 2022

(51) Int. Cl. G16H 10/60

(52) U.S. Cl. CPC G16H 10/60

(57) ABSTRACT

A system and method for integrating health related data is disclosed. The system and method can receive a query for health data. Based on the query can determine what type of health data the query requests. Based on determining what type of health data the query requests the data may be retrieved from the appropriate source. Once the data is retrieved, the system can transmit the retrieved data to an application on a client device in response to the query.

### TECHNICAL FIELD

Aspects relate to a system, architecture, and methods that integrate various health related data, specifically a system that integrates standardized and non-standardized health related data.

### BACKGROUND

The Fast Healthcare Interoperability Resources (FHIR) standard is a healthcare data exchange standard describing and defining data formats for exchanging electronic health related data. The goal of FHIR is to facilitate interoperation between legacy healthcare systems and Electronic Health Records (EHR) systems. FHIR makes it easy to provide health related data to healthcare providers and individuals on a wide variety of devices, from computers to tablets to cell phones, and to allow software application developers to provide custom software applications to supplement the functioning of EHR systems by using standard data formats to represent health related data. By defining standard data formats, FHIR allows software applications to easily integrate into existing systems to exchange data.

Use of the FHIR standard, however, is not always applicable when developing custom software applications. This is because FHIR does not provide standard definitions/formats for every possible type of health related data. Thus, software application developers sometimes have to define custom data objects and/or data structures to represent certain health information not included in the FHIR standard. An example of this is prescription order numbers, which the FHIR standard does not have a defined standard/format for. Thus, a software application developer creating a software application related to prescription orders will have to define their own format and/or way of representing prescription orders. The same issue exists for any other custom or non-standard health related data.

Software application developers may also sometimes partially implement FHIR into their applications, thus leaving their applications not fully operable or compliant with other FHIR based systems. This may be because the applications they develop may only deal with certain types of health related data, and thus the applications may be implemented to recognize only those particular FHIR data formats, while excluding other FHIR data formats. As a result, the applications may not be able to ingest a complete FHIR data set.

Another issue related to conventional systems trying to integrate FHIR and non-FHIR data is that traditionally, the approach has been to replicate the FHIR data across multiple systems in different formats to integrate it with applications using non-FHIR data. This brings in challenges as the data is never consistent if data is updated later on because once replicated the data is no longer synchronized with the source it was obtained from.

Thus, systems and methods are needed to address these issues, and provide a way to integrate FHIR and non-FHIR data so that the most up to date data may be ingested and used in software applications.

### SUMMARY

Aspects of this disclosure are directed to a system and methods for integrating health related data. Specifically, the system and methods provide a platform that enables software applications to query and ingest both FHIR and non-FHIR data formats. The system and methods also allow software developers to define custom healthcare data types combining both FHIR and non-FHIR data types and to retrieve from both FHIR and non-FHIR sources the data to combine as the custom data type. Throughout this disclosure FHIR data and non-FHIR data may also be referred to as a “FHIR resource” or “non-FHIR resource.” The term “resource” is interchangeable with “data” or “data type.”

In aspects, the system and methods can operate using one or more computing devices. In aspects, the operation may begin by having the one or more computing devices receive, from an application on a client device, a query for health data. The system can determine whether the query requests health data that is: a FHIR resource, a non-FHIR resource, or a custom resource. The custom resource may be health data comprising a combination of the FHIR resource and the non-FHIR resource. In aspects, if determined that the query requests health data that is the FHIR resource, the system can retrieve the FHIR resource from a FHIR server via a GraphQL query or via FHIR native REST API. In aspects, if determined that the query requests health data that is a non-FHIR resource, the system can retrieve the non-FHIR resource from a non-FHIR server. The retrieval may be done using a GraphQL query, Structured Query Language (SQL) queries, and/or via Representational State Transfer (REST) application programming interface (API) calls. In aspects, if determined that the query requests health data that is the custom resource, the system can further access a metadata table, where the metadata table defines a relationship between the FHIR resource and the non-FHIR resource for the custom resource, and based on the defined relationship, retrieve the FHIR resource from the FHIR server using the GraphQL query or via FHIR native REST API, and further retrieve the non-FHIR resource. In aspects, the metadata table refers to a data structure or data object that may be dynamically configurable to define the structures of the FHIR resource and the non-FHIR resource and the relationship between the FHIR resource and the non-FHIR resource for the custom resource. The definition can map non-FHIR data and FHIR data together to create custom health data types that may be joined, so that they may be retrieved and combined to obtain the custom resource. In aspects, once both the FHIR and non-FHIR data are retrieved, the system can combine the retrieved FHIR resource and the non-FHIR resource as the custom resource. In aspects, once the data is retrieved, the system can transmit the retrieved FHIR resource, non-FHIR resource, or custom resource to the application on the client device in response to the query. The data can then be used by the application to perform whatever function the application was designed for.

In aspects, the system and methods can operate in real-time. For example, the system can transmit the retrieved FHIR resource, non-FHIR resource, or custom resource to the client device in real-time from when the query is received.

In aspects, the system may be implemented in a cloud computing environment, using a cloud computing service. For example, the various servers and computing devices that make up the system, and operate to provide the system its functionality, may be part of a cloud computing environment.

In aspects, the system can further format the retrieved FHIR resource, non-FHIR resource, or custom resource in a JavaScript Object Notation (JSON) file, and transmit the JSON file to the application on the client device in response to the query.

In aspects, the system can further receive, from the application on the client device, a request to update the FHIR resource, the non-FHIR resource, or the custom resource. If the request is to update the FHIR resource, the system can update the FHIR resource on the FHIR server. If the request is to update the non-FHIR resource, the system can update the non-FHIR resource on the non-FHIR server. If the request is to update the custom resource, the system can update the FHIR resource of the custom resource on the FHIR server and the non-FHIR resource of the custom resource on the non-FHIR server.

In aspects, the system can further authenticate the application or the client device prior to determining whether the query requests health data that is: the FHIR resource, the non-FHIR resource, or the custom resource. In aspects, the system, as part of the authentication can determine if the application or the client device has permission to access the health data. Additionally, the system can further perform an authorization to determine whether a user of the application or the client device has permission to access the health data even if the application or the client device is authenticated. This can be, for example, based on a user's credentials or account information indicating they are authorized to view the data (e.g., a doctor will only be able to see health data for only his own patients as opposed to other doctor's patients from the same clinic or hospital).

Certain aspects of the disclosure have other steps or elements in addition to or in place of those mentioned above. The steps or elements will become apparent to those skilled in the art from a reading of the following detailed description when taken with reference to the accompanying drawings.

### BRIEF DESCRIPTION OF THE DRAWINGS

The accompanying drawings, which are incorporated herein and form a part of the specification, illustrate aspects of the present disclosure and, together with the description, further serve to explain the principles of the disclosure and to enable a person skilled in the art to make and use the aspects.

FIG. 1 is an example system for integrating health related data, according to aspects.

FIG. 2 is an example method of operating the system, according to aspects.

FIG. 3 is an example architecture of the components implementing the system, according to aspects.

In the drawings, like reference numbers generally indicate identical or similar elements. Additionally, generally, the left-most digit(s) of a reference number identifies the drawing in which the reference number first appears.

### DETAILED DESCRIPTION

The following aspects are described in sufficient detail to enable those skilled in the art to make and use the disclosure. It is to be understood that other aspects are evident based on the present disclosure, and that system, process, or mechanical changes may be made without departing from the scope of an aspect of the present disclosure.

In the following description, numerous specific details are given to provide a thorough understanding of aspects. However, it will be apparent that aspects may be practiced without these specific details. To avoid obscuring an aspect, some well-known circuits, system configurations, and process steps are not disclosed in detail.

The drawings showing aspects of the system are semi-diagrammatic, and not to scale. Some of the dimensions are for the clarity of presentation and are shown exaggerated in the drawing figures. Similarly, although the views in the drawings are for ease of description and generally show similar orientations, this depiction in the figures is arbitrary for the most part. Generally, the system may be operated in any orientation.

Certain aspects have other steps or elements in addition to or in place of those mentioned. The steps or elements will become apparent to those skilled in the art from a reading of the following detailed description when taken with reference to the accompanying drawings.

### System Overview and Function

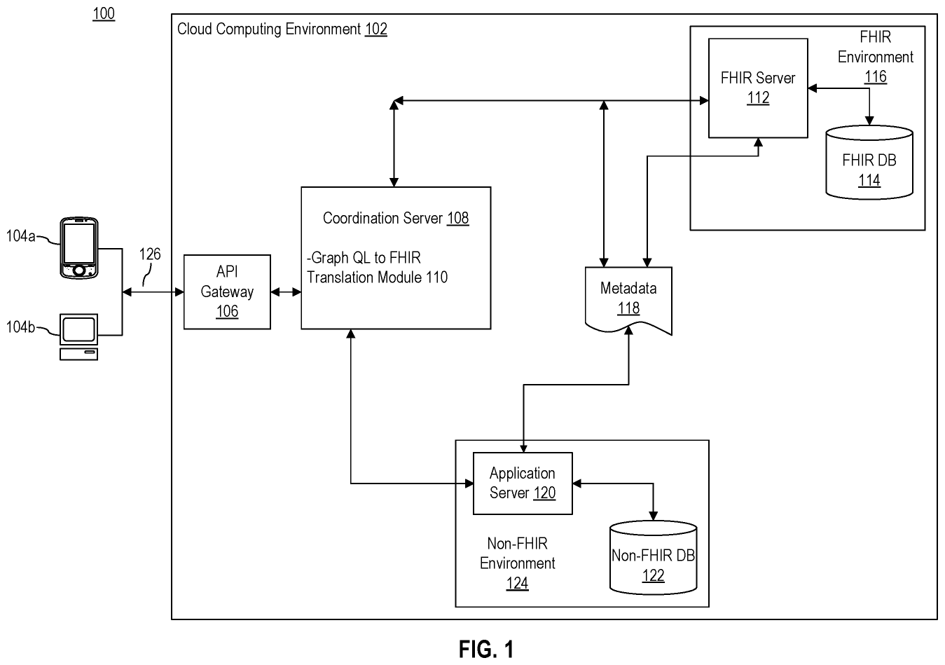

FIG. 1 is an example system 100 for integrating health related data, according to aspects. In aspects, the system 100 may be implemented using one or more client devices 104, which may be coupled to one or more servers via a network 126. FIG. 1 shows two client devices {104a and 104b}. This is exemplary and any number of client devices may be used. The client devices 104 may be computing devices used by patients, healthcare providers, pharmacies, etc. to retrieve or access health related data. The servers shown in FIG. 1 include a coordination server 108, a FHIR server 112, and an application server 120. Other servers may be implemented to facilitate the functioning of the system 100. How the client devices 104 and the servers (108, 112, and 120) function within the framework of the system 100 will be described further below.

The client devices 104 may be any of a variety of centralized or decentralized computing devices. For example, the client devices 104 may be mobile devices (e.g., a cell phone, a smartphone, a tablet, etc.), laptop computers, or desktop computers. While the client devices 104 can couple with the network 126 to communicate with the servers, the client devices 104 can also function as stand-alone devices separate from the servers. Stand-alone refers to a device being able to work and operate independently of other devices.

In aspects, the client devices 104 can have a software application installed thereon. The software application may be a healthcare related application where a patient or healthcare provider can, for example, view, edit, or access health related data. As examples, the healthcare related application can include a patient portal connecting a patient to a healthcare provider, an application where a patient can view health related data, a health insurance application, an application where a physician can order prescription medications, etc. These are merely examples of what the healthcare related application may be and are not meant to be limiting. The software application may be installed on the client devices 104 as instructions on a non-transitory computer readable medium. The client devices 104 can execute the instructions to perform whatever functionality the software application was designed to provide. In other aspects, the client devices 104 and the servers can have portions of the healthcare related application installed as instructions on a non-transitory computer readable medium of each. Thus, the client devices 104 and the servers can both execute portions of the software to implement the functionality of the healthcare related application using client-server architectures.

In aspects, the servers can also be a variety of centralized or decentralized computing devices. For example, the servers may be implemented using a mobile device, a laptop computer, a desktop computer, grid-computing devices, virtualized computing devices, cloud computing devices, peer-to-peer distributed computing devices, a server farm, or a combination thereof. The servers may be centralized in a single room, distributed across different rooms, distributed across different geographic locations, or embedded within the network 126. While the servers can couple with the network 126 to communicate with the client devices 104, the servers can also function as stand-alone devices separate from the client devices 104. In aspects, the cloud computing devices may be part of a cloud computing environment 102. The cloud computing environment 102 may be a public or private cloud service. Examples of a public cloud include Amazon Web Services (AWS), IBM Cloud, Oracle Cloud Solutions, Microsoft Azure Cloud, and Google Cloud. A private cloud refers to a cloud environment similar to a public cloud with the exception that it is operated solely for a single organization.

The network 126 refers to a telecommunications network, such as a wired or wireless network. The network 126 can span and represent a variety of networks and network topologies. For example, the network 126 can include wireless communication, wired communication, optical communication, ultrasonic communication, or a combination thereof. For example, satellite communication, cellular communication, Bluetooth, Infrared Data Association standard (IrDA), wireless fidelity (WiFi), and worldwide interoperability for microwave access (WiMAX) are examples of wireless communication that may be included in the network 126. Cable, Ethernet, digital subscriber line (DSL), fiber optic lines, fiber to the home (FTTH), and plain old telephone service (POTS) are examples of wired communication that may be included in the network 126. Further, the network 126 can traverse a number of topologies and distances. For example, the network 126 can include a direct connection, personal area network (PAN), local area network (LAN), metropolitan area network (MAN), wide area network (WAN), or a combination thereof. For illustrative purposes, in the aspect of FIG. 1, the system 100 is shown with the client devices 104 and servers as end points of the network 126. This, however, is exemplary and it is understood that the system 100 can have a different partition between the client devices 104, the servers, and the network 126. For example, the client devices 104 and the servers can also function as part of the network 126.

How the system 100 operates will now be described. In aspects, the system 100 may begin its operation when a query is received by one of the servers of the system 100, from the healthcare related application installed on a client device (e.g., 104a or 104b). The query may be for health data. The query may be a result of the healthcare related application needing health related data to perform an operation (e.g., obtaining patient health information, obtaining lab results, obtaining health charts and doctor/patient meeting notes, filling a prescription, etc.). In aspects, the query may be transmitted to one or more servers of the system 100 via the network 126. In aspects, the query may be made via an application programming interface (API) call to a server of the system 100. In aspects, the API call can initially be received by an API gateway 106.

The API gateway 106 refers to an API management tool that sits between the client devices 104 and the servers. The API gateway 106 can accept all API calls to the system 100, aggregate the various services required to fulfill them, and return the appropriate result back to the client devices 104. In aspects, the API gateway 106 can, either by itself or in conjunction with other servers of the system 100 (e.g., the coordination server 108), function to add a layer of security for the system 100. For example, the API gateway 106 by itself or in conjunction with other servers, can authenticate whether the query is coming from an authorized source, by for example authenticating the healthcare related application and/or the client device from which the query is originating from. The authentication may be done using any known authentication techniques known in the art, such as two-factor authentication, token authentication, public-private key based authentication, etc. In aspects, the system 100, as part of the authentication, can determine if the application or the client device has permission to access the health data. This can be done by, for example, determining, based on security settings or credentials of the application or the client device, whether the application or the client device can access the data. These security settings or credentials can indicate, for example, that an application or client device is on a blocked list, has limited read, write, or editing access, can access certain categories of data, or has full access to the data. Additionally, the system can further perform an authorization to determine whether a user of the application or the client device has permission to access the health data even if the application or the client device is authenticated. This can be, for example, based on a user's credentials or account information indicating they are authorized to view the data (e.g., a doctor will only be able to see health data for only his own patients as opposed to other doctor's patients from the same clinic or hospital). In aspects, the API gateway 106 can also facilitate data and protocol translations where necessary to ensure intended information is exchanged between the client devices 104 and the servers.

In aspects, once the query is received by the API gateway 106 and/or is authenticated and permitted to be transmitted to the servers of the system 100, control and the query can pass to the coordination server 108. The coordination server 108 enables the coordination of the data retrieval functions for the system 100. In aspects, the coordination server 108, upon receipt of the query, can determine what type of health data the query is requesting. In aspects, the determination functions may be performed by one or more modules stored on a non-transitory computer readable medium of the coordination server 108. In aspects, the coordination server 108 can execute the instructions to perform the determination functions. For example, the coordination server 108 can determine whether the query is requesting FHIR data or non-FHIR data to be retrieved. This may be done in a variety of ways. For example, in aspects, the coordination server 108 can look at the names or descriptions of the data fields requested via the query, and map the names against names for known FHIR data types. If a match is found, the coordination server 108 can determine that a FHIR data type is requested, and can proceed to retrieve the FHIR data from servers or databases in a FHIR environment 116. If a match is not found, the coordination server 108 can determine that a non-FHIR data type is requested, and can proceed to retrieve the non-FHIR data from servers or database in a non-FHIR environment 124. In aspects, in addition to or in lieu of looking at the names or descriptions of data fields requested, a further way to determine the type of data the query is requesting is to look for a flag, parameter, or variable that may be included in an API call with the query, and based on the flag, parameter, or variable, determine whether the data to be retrieved is a FHIR data type or a non-FHIR data type. In aspects, the flag, parameter, or variable can, for example, indicate that the data being requested is a FHIR data or a non-FHIR data.

The FHIR environment 116 refers to a computing environment or cluster of computers within a computing environment that implement the FHIR data standard and/or store FHIR data/resources. In aspects, the FHIR environment 116 may be a part of the cloud computing environment 102 or be external to the cloud computing environment 102. In aspects, the FHIR environment 116 can include a FHIR server 112 and a FHIR database 114.

The non-FHIR environment 124 refers to a computing environment or cluster of computers within a computing environment that implement the non-FHIR data standard and/or store non-FHIR data/resources. In aspects, the non-FHIR environment 124 may be a part of the cloud computing environment 102 or be external to the cloud computing environment 102. In aspects, the non-FHIR environment 124 can include the application server 120 and a non-FHIR database 122. The application server 120 is as a non-FHIR server.

In aspects, if retrieving data from the FHIR environment 116, the coordination server 108 can forward the request for the data to the FHIR server 112. The FHIR can receive requests from the coordination server 108 and coordinate the retrieval of the FHIR data from within the FHIR environment 116. In aspects, the FHIR database 114 can store the FHIR data and from where the FHIR server 112 can retrieve the FHIR data. In aspects, the coordination server 108 can make an API call to the FHIR server 112 using a GraphQL query or FHIR native REST API to retrieve the FHIR data. To facilitate the use of GraphQL, the coordination server 108 can have a GraphQL to FHIR translation module 110 implement code or instructions that can communicate the request for data made by the query, and generate a GraphQL query to the FHIR server 112 to retrieve the FHIR data requested. GraphQL is an open-source data query and manipulation language for APIs. A person skilled in the art (POSA) will know how to implement GraphQL and how to implement API calls and queries using the same. Therefore, a detailed description of GraphQL will not be provided in this disclosure. It is assumed that a GraphQL query may be made to retrieve the FHIR data.

Using GraphQL to retrieve the FHIR data has several benefits. First, it allows for the retrieval of targeted health related data, thus reducing data overhead when retrieving the FHIR data. This is because many FHIR data types have sub-data fields/resources. For example the FHIR standard format for how to represent a patient within a software application, includes many sub-fields indicating specific formats for how to represent the patient's name, contact details, gender, etc. If using conventional technologies like REST API calls to retrieve the FHIR data, all the sub-data fields/resources will be returned and a further step of having to filter out the unwanted data from the returned data set will have to be performed to get the specific data requested. This is because APIs implemented using technologies like the REST architecture and methodology only have the ability to return bulk data and cannot target specific sub-fields of the FHIR data to return. The exception being the use of FHIR native REST API, which is specific to FHIR and can also return specific data fields. Thus, if an application only wants the patient's name returned, using conventional REST APIs, they will not only get the patients name but all information regarding the patient's contact details, gender, etc., that are also part of the FHIR standard for how to represent a patient. Most of this information will not be needed and results in excess data being transferred. This can cause a huge data overhead when an application or multiple applications are trying to retrieve data from the FHIR environment 116. As a result, the latency increases and speed at which data is retrieved slows down. Thus, by having the system 100 implement GraphQL to retrieve the FHIR data, specific fields for each FHIR data may be returned for the query without the excess data, thus reducing network traffic, latency, and overhead. Thus, system 100 can retrieve data more quickly than conventional systems. This architecture results in an improved overall speed of data retrieval than conventional systems, thus improving computer functionality.

Continuing with the example, in aspects, if the coordination server 108 determines that non-FHIR data is to be returned, the coordination server 108 can proceed to retrieve the non-FHIR data from the non-FHIR environment 124. In aspects, the coordination server 108 can send requests for the requested data from the query to the application server 120. The application server 120 can receive requests and coordinate the retrieval of the non-FHIR data from within the non-FHIR environment 124. In aspects, the data may be retrieved from the non-FHIR database 122, which can store the non-FHIR data. The data retrieval from the non-FHIR environment does not have to use GraphQL queries. In aspects, when coordinating the retrieval of non-FHIR data from the non-FHIR environment 124, the coordination server 108 can make an API call to the non-FHIR environment 116 (and more particularly to the application server 120) using a GraphQL query, SQL queries, or via REST API calls to retrieve the FHIR data/resource. This is because typically the application server 120 does not need to handle as many requests as the FHIR server 112 and the non-FHIR data typically does not have as many sub-fields (if any) as FHIR data, and therefore the latency and data overhead concerns when retrieving data is less.

In aspects, the query can also request a custom data. The custom data refers to a health related data that comprises a combination of the FHIR resource and the non-FHIR resource. Custom data may be defined by a developer or administrator of the healthcare related application. Custom data objects may be defined to represent health care data that combines FHIR data and/or non-FHIR data. As an example, a custom data object may be defined in a healthcare related application for a “Report.” In aspects, the “Report” can include the name of a patient and a prescription order number of the patient. A POSA will recognize that the FHIR standard defines a standard format to represent the name of the patient but not a prescription order number. Therefore, the custom data object of a “Report” can have both a FHIR data component and a non-FHIR data component. In aspects, if the coordination server 108 determines that the type of data requested is a custom data (using the same methodologies described previously using data names, descriptions, flags, etc.), the coordination server 108 can take further steps to retrieve the data. In aspects, the coordination server 108 can proceed to retrieve the data by accessing a metadata table 118. The metadata table 118 refers to a data structure or data object that may be dynamically configurable to define the relationship between the FHIR data and the non-FHIR data for the custom resource. The definition can map non-FHIR data and FHIR data together to create custom health data types that may be joined, so that they may be retrieved and combined to obtain the custom resource. Dynamically configurable refers to the metadata table 118 being modifiable by a developer or administrator of the system 100 such that these relationships may be defined or modified, by adding, deleting, or adjusting relationships between the FHIR data and/or the non-FHIR data.

In aspects, the coordination server 108 can access the metadata table 118 and do a table lookup or otherwise determine what the particular FHIR data and/or non-FHIR data the custom data object comprises, such that the data may be retrieved based on the defined relationship. For example, and taking the example of the custom data object of “Report,” the metadata table 118 can indicate that a “Report” object comprises the name of a patient and a prescription order number of the patient. In aspects, based on the defined relationship, the coordination server 108 can send a request to both the FHIR server 112 and the application server 120 to retrieve the FHIR resource from the FHIR server using the GraphQL query or using FHIR native REST API, and the non-FHIR resource using any of the techniques previously indicated. In aspects, once the data is retrieved, the FHIR and the non-FHIR data may be returned to the coordination server 108 so that it may be combined as the custom data.

In aspects, once any of the data requested, whether it be the FHIR data, the non-FHIR data, or the custom data is retrieved, the data can then be transmitted to the coordination server 108 which can aggregate the data and/or transmit it back to the client device(s) 104 and/or application installed on the client devices 104 that requested the data. In aspects, the coordination server 108 can format the data before it is transmitted back to the client devices 104 and/or application. For example, the coordination server 108 can format the retrieved data in a JSON file, an XML file, or any other text based file format prior to transmitting it to the client devices 104 and/or application. The formatting can assist the client devices 104 and/or application recognize and extract the data once it is received. In aspects, the transmittal may be done in real-time. Real-time refers to transmitting the retrieved data within seconds or milliseconds from when the query is received by the coordination server 108.

In aspects, and in addition to having the ability to query for data, the system 100 can allow for FHIR data, non-FHIR data, or custom data to be modified. Thus, the system 100 can function bi-directionally so that data may be written to or updated on the FHIR server 112, application server 120, or a combination thereof. In aspects, the modification can result from the application installed on the client devices 104 making a request to modify data. This may be due to, for example, a user, a healthcare provider, etc. updating health related data via the application. In aspects, a request to update the FHIR data, the non-FHIR data, or the custom data may be received from the application on a client device (e.g., 104a or 104b). In aspects, if the request is to update the FHIR data, the coordinator server 108 can transmit a further request and the updated values for the data to the FHIR server 112, which can update the FHIR data via the FHIR server 112, on for example the FHIR database 114. In aspects, the further request may be via an API call to the FHIR server 112 with the updated values of the data to be updated.

In aspects, if the request is to update the non-FHIR data, the coordination server 108 can transmit a further request and the updated values for the data to the application server 120, which can update the non-FHIR data via the application server 120, on for example the non-FHIR database 122. In aspects, the further request may be via an API call to the application server 120 with the updated values of the data to be updated.

In aspects, if the request is to update the custom resource, the coordinator server 108 can access the metadata table 118 to determine what FHIR and/or non-FHIR data needs to be updated. Based on referencing the metadata table 118, the coordinator server 108 can transmit a further request and the updated values for the data to either the FHIR server 112, application server 120, or a combination thereof to update the FHIR data component of the custom data via the FHIR server 112, and the non-FHIR data portion of the custom resource on the application server 120.

The modules described in FIG. 1 may be implemented as instructions stored on a non-transitory computer readable medium to be executed by one or more computing units such as a processor, a special purpose computer, an integrated circuit, integrated circuit cores, or a combination thereof. The non-transitory computer readable medium may be implemented with any number of memory units, such as a volatile memory, a nonvolatile memory, an internal memory, an external memory, or a combination thereof. The non-transitory computer readable medium may be integrated as a part of any of the servers or devices of the system 100 or installed as a removable portion of the servers or devices of the system 100.

It has been discovered that the system 100 described above significantly improves the state of the art from conventional systems because it provides an architecture in which FHIR and non-FHIR data may be seamlessly retrieved and/or modified on a single platform. Typically healthcare platforms are built either on FHIR or non-FHIR system backends and architectures, and therefore have a difficult time integrating healthcare data that does not fit within the standards and/or definitions of their architectures. By using system 100, application developers can develop applications that can integrate both FHIR and non-FHIR data more easily. The novelty stems at least from the use of the coordinator server 108 in conjunction with the metadata table 118, which may be used to define relationships between FHIR and non-FHIR data and map/join these two types of data. Additionally, the mapping and joining allows application developers to define custom data types and to easily retrieve and/or update these data types using a single platform, interface, and API. This adds the ability for application developers to customize healthcare applications using a full range of FHIR data and non-FHIR data in a way that is not currently possible.

Another improvement provided by the system 100 is that it always retrieves the most up to date FHIR and non-FHIR data. This is because it retrieves the data directly from the environment in which it is stored (i.e., the FHIR environment 116 and/or the Non-FHIR environment 124). Conventionally when applications attempt to integrate FHIR and non-FHIR data, applications have to download all the FHIR and/or non-FHIR data to their local environments or servers on which they run (i.e., an application server), and then have to parse and process the data to obtain the data that the application needs. The system 100, however, allows for applications to retrieve any type of data it needs directly from the environment in which it is stored, through the use of the coordination server 108 and the metadata table 118. This has the added benefit of the application always processing data that is up to date, because in conventional systems there could be a situation where data is downloaded but is later updated, resulting in a data mismatch problem.

A further improvement is that the system 100 provides for better speed and lower latency when retrieving FHIR data. As indicated, FHIR data can have many sub-fields for any particular data type. By using GraphQL queries, the system 100 can retrieve targeted FHIR data rather than the entire data set/fields for a data type. This is because GraphQL queries allow for targeted retrieval of data, whereas conventional systems using REST API calls will receive data dumps of the entire FHIR data type. This is because REST API architectures are not designed to retrieve specific fields of data.

### Methods of Operation

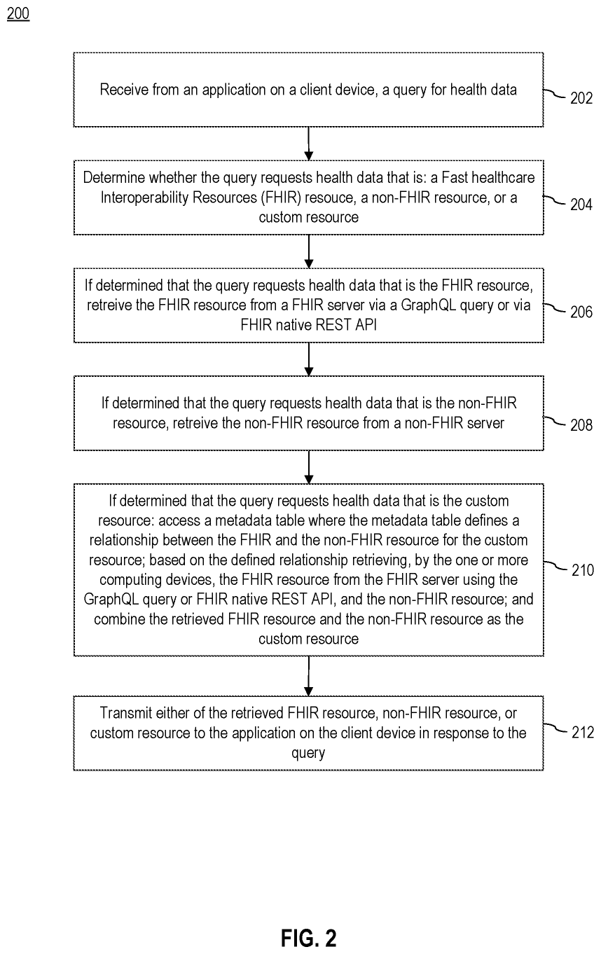

FIG. 2 is an example method 200 of operating the system 100, according to aspects. The method 200 may be performed by any of the servers or devices of the system 100, for example the coordination server 108. Method 200 includes receiving, from an application on a client device (e.g., 104a or 104b), a query for health data, as shown in 202. The system 100 can then determine whether the query requests health data that is: a FHIR resource, a non-FHIR resource, or a custom resource, as shown in 204. If determined that the query requests health data that is the FHIR resource, the FHIR resource may be retrieved from the FHIR server 112 via a GraphQL query or via FHIR native REST API, as shown in 206. If determined that the query requests health data that is the non-FHIR resource, the non-FHIR resource may be retrieved from a non-FHIR server, for example the application server 120, as shown in 208. In aspects, the retrieval may be via a GraphQL query, a SQL query, and/or a REST API call. If determined that the query requests health data that is the custom resource, the system 100 can access a metadata table 118 that defines a relationship between the FHIR resource and the non-FHIR resource for the custom resource, and based on the defined relationship retrieve the FHIR resource from the FHIR server using the GraphQL query or FHIR native REST API, and the non-FHIR resource via any previously indicated technique. Once retrieved, the data may be combined as the custom resource, as shown in 210. The retrieved FHIR resource, non-FHIR resource, or custom resource can then be transmitted to the application on the client device (e.g., 104a or 104b) in response to the query, as shown in 212.

The operation of method 200 is performed, for example, by system 100, in accordance with aspects described above. In aspects, the method 200 may be performed in real-time, as described with respect to FIG. 1.

### Components of the System

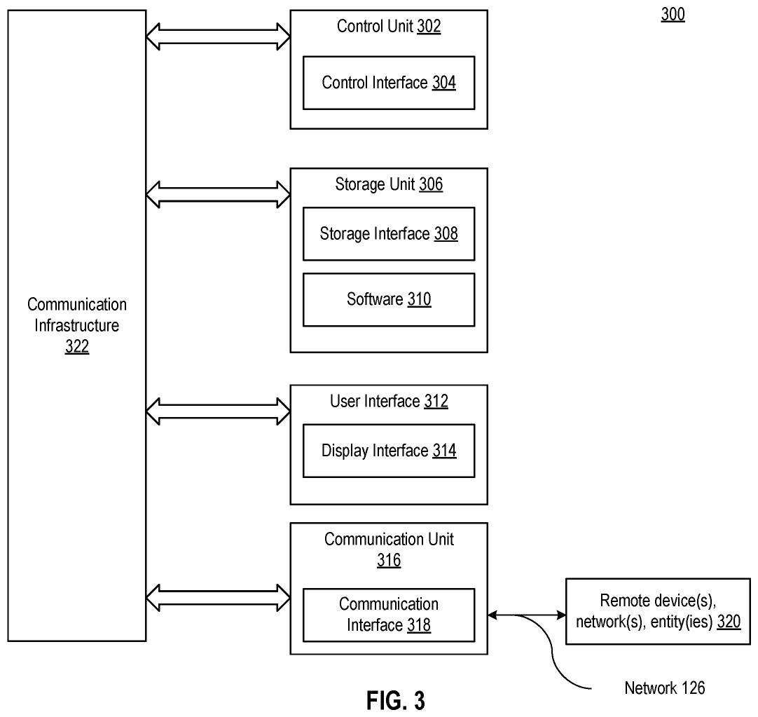

FIG. 3 is an example architecture 300 of the components implementing the system 100, according to aspects. In aspects, the components may be the components of the servers or client devices 104 that are used to implement the functions of the system 100. In aspects the components may include a control unit 302, a storage unit 306, a communication unit 316, and a user interface 312. The control unit 302 may include a control interface 304. The control unit 302 may execute a software 310 to provide some or all of the intelligence of system 100. The control unit 302 may be implemented in a number of different ways. For example, the control unit 302 may be a processor, an application specific integrated circuit (ASIC), an embedded processor, a microprocessor, a hardware control logic, a hardware finite state machine (FSM), a digital signal processor (DSP), a field programmable gate array (FPGA), or a combination thereof.

The control interface 304 may be used for communication between the control unit 302 and other functional units or devices of system 100. The control interface 304 may also be used for communication that is external to the functional units or devices of system 100. The control interface 304 may receive information from the functional units or devices of system 100, or from remote devices 320, such as those of EHRS systems of hospitals or patient care providers that maybe used in conjunction with the system 100, or may transmit information to the functional units or devices of system 100, or to remote devices 320. The remote devices 320 refer to units or devices external to system 100.

The control interface 304 may be implemented in different ways and may include different implementations depending on which functional units or devices of system 100 or remote devices 320 are being interfaced with the control unit 302. For example, the control interface 304 may be implemented with a pressure sensor, an inertial sensor, a microelectromechanical system (MEMS), optical circuitry, waveguides, wireless circuitry, wireline circuitry to attach to a bus, an application programming interface, or a combination thereof. The control interface 304 may be connected to a communication infrastructure 322, such as a bus, to interface with the functional units or devices of system 100 or remote devices 320.

The storage unit 306 may store the software 310. For illustrative purposes, the storage unit 306 is shown as a single element, although it is understood that the storage unit 306 may be a distribution of storage elements. Also for illustrative purposes, the storage unit 306 is shown as a single hierarchy storage system, although it is understood that the storage unit 306 may be in a different configuration. For example, the storage unit 306 may be formed with different storage technologies forming a memory hierarchical system including different levels of caching, main memory, rotating media, or off-line storage. The storage unit 306 may be a volatile memory, a nonvolatile memory, an internal memory, an external memory, or a combination thereof. For example, the storage unit 306 may be a nonvolatile storage such as nonvolatile random access memory (NVRAM), Flash memory, disk storage, or a volatile storage such as static random access memory (SRAM) or dynamic random access memory (DRAM).

The storage unit 306 may include a storage interface 308. The storage interface 308 may be used for communication between the storage unit 306 and other functional units or devices of system 100. The storage interface 308 may also be used for communication that is external to system 100. The storage interface 308 may receive information from the other functional units or devices of system 100 or from remote devices 320, or may transmit information to the other functional units or devices of system 100 or to remote devices 320. The storage interface 308 may include different implementations depending on which functional units or devices of system 100 or remote devices 320 are being interfaced with the storage unit 306. The storage interface 308 may be implemented with technologies and techniques similar to the implementation of the control interface 304.

The communication unit 316 may enable communication to devices, components, modules, or units of system 100 or to remote devices 320. For example, the communication unit 316 may permit the system 100 to communicate between its components the client devices 104 and servers. The communication unit 316 may further permit the devices of system 100 to communicate with remote devices 320 such as an attachment, a peripheral device, or a combination thereof through the network 126.

As previously indicated, the network 126 may span and represent a variety of networks and network topologies. For example, the network 126 may be a part of a network and include wireless communication, wired communication, optical communication, ultrasonic communication, or a combination thereof. For example, satellite communication, cellular communication, Bluetooth, Infrared Data Association standard (IrDA), wireless fidelity (WiFi), and worldwide interoperability for microwave access (WiMAX) are examples of wireless communication that may be included in the network 126. Cable, Ethernet, digital subscriber line (DSL), fiber optic lines, fiber to the home (FTTH), and plain old telephone service (POTS) are examples of wired communication that may be included in the network 126. Further, the network 126 may traverse a number of network topologies and distances. For example, the network 126 may include direct connection, personal area network (PAN), local area network (LAN), metropolitan area network (MAN), wide area network (WAN), or a combination thereof.

The communication unit 316 may also function as a communication hub allowing system 100 to function as part of the network 126 and not be limited to be an end point or terminal unit to the network 126. The communication unit 316 may include active and passive components, such as microelectronics or an antenna, for interaction with the network 126.

The communication unit 316 may include a communication interface 318. The communication interface 318 may be used for communication between the communication unit 316 and other functional units or devices of system 100 or to remote devices 320. The communication interface 318 may receive information from the other functional units or devices of system 100, or from remote devices 320, or may transmit information to the other functional units or devices of the system 100 or to remote devices 320. The communication interface 318 may include different implementations depending on which functional units or devices are being interfaced with the communication unit 316. The communication interface 318 may be implemented with technologies and techniques similar to the implementation of the control interface 304.

The user interface 312 may present information generated by system 100. In aspects, the user interface 312 allows a developer or an administrator of the system 100 to interface with the system 100. The developer or administrator can, for example, update or define definitions between FHIR data and non-FHIR data for the metadata table 118 using the user interface 312. The user interface 312 may include an input device and an output device. Examples of the input device of the user interface 312 may include a keypad, buttons, switches, touchpads, soft-keys, a keyboard, a mouse, or any combination thereof to provide data and communication inputs. Examples of the output device may include a display interface 314. The control unit 302 may operate the user interface 312 to present information generated by system 100. The control unit 302 may also execute the software 310 to present information generated by system 100, or to control other functional units of system 100. The display interface 314 may be any graphical user interface such as a display, a projector, a video screen, or any combination thereof.

The terms “module” or “unit” referred to in this disclosure can include software, hardware, or a combination thereof in an aspect of the present disclosure in accordance with the context in which the term is used. For example, the software may be machine code, firmware, embedded code, or application software. Also for example, the hardware may be circuitry, a processor, a special purpose computer, an integrated circuit, integrated circuit cores, a pressure sensor, an inertial sensor, a microelectromechanical system (MEMS), passive devices, or a combination thereof. Further, if a module or unit is written in the system or apparatus claims section below, the module or unit is deemed to include hardware circuitry for the purposes and the scope of the system or apparatus claims.

The modules and units in the aforementioned description of the aspects may be coupled to one another as described or as shown. The coupling may be direct or indirect, without or with intervening items between coupled modules or units. The coupling may be by physical contact or by communication between modules or units.

The above detailed description and aspects of the disclosed system 100 are not intended to be exhaustive or to limit the disclosed system 100 to the precise form disclosed above. While specific examples for system 100 are described above for illustrative purposes, various equivalent modifications are possible within the scope of the disclosed system 100, as those skilled in the relevant art will recognize. For example, while processes and methods are presented in a given order, alternative implementations may perform routines having steps, or employ systems having processes or methods, in a different order, and some processes or methods may be deleted, moved, added, subdivided, combined, or modified to provide alternative or sub-combinations. Each of these processes or methods may be implemented in a variety of different ways. Also, while processes or methods are at times shown as being performed in series, these processes or blocks may instead be performed or implemented in parallel, or may be performed at different times.

The resulting method 200 and system 100 is cost-effective, highly versatile, and accurate, and may be implemented by adapting components for ready, efficient, and economical manufacturing, application, and utilization. Another important aspect of the present disclosure is that it valuably supports and services the historical trend of reducing costs, simplifying systems, and/or increasing performance.

These and other valuable aspects of the present disclosure consequently further the state of the technology to at least the next level. While the disclosed aspects have been described as the best mode of implementing system 100, it is to be understood that many alternatives, modifications, and variations will be apparent to those skilled in the art in light of the descriptions herein. Accordingly, it is intended to embrace all such alternatives, modifications, and variations that fall within the scope of the included claims. All matters set forth herein or shown in the accompanying drawings are to be interpreted in an illustrative and non-limiting sense.

What is claimed is:

1. A computer-implemented method for integrating health related data, the method comprising:
receiving, by one or more computing devices and from an application on a client device, a query for health data;
determining, by the one or more computing devices, whether the query requests health data that is:
a Fast Healthcare Interoperability Resources (FHIR) resource,
a non-FHIR resource, or
a custom resource, wherein the custom resource is health data comprising a combination of the FHIR resource and the non-FHIR resource;
if determined that the query requests health data that is the FHIR resource, retrieving, by the one or more computing devices, the FHIR resource from a FHIR server via a GraphQL query or via a FHIR native REST API;
if determined that the query requests health data that is the non-FHIR resource, retrieving, by the one or more computing devices, the non-FHIR resource from a non-FHIR server;
if determined that the query requests health data that is the custom resource:
accessing, by the one or more computing devices, a metadata table, wherein the metadata table defines a relationship between the FHIR resource and the non-FHIR resource for the custom resource,
based on the defined relationship retrieving, by the one or more computing devices, the FHIR resource from the FHIR server using the GraphQL query or the FHIR native REST API, and the non-FHIR resource, and
combining the retrieved FHIR resource and the non-FHIR resource as the custom resource; and
transmitting, by the one or more computing devices, the retrieved FHIR resource, non-FHIR resource, or custom resource to the application on the client device in response to the query.

2. The method of claim 1, further comprising transmitting, by the one or more computing devices, the retrieved FHIR resource, non-FHIR resource, or custom resource to the client device in real-time from when the query is received.

3. The method of claim 1, wherein the method is implemented using a cloud computing service.

4. The method of claim 1, further comprising:
formatting, by the one or more computing devices, the retrieved FHIR resource, non-FHIR resource, or custom resource in a JSON file; and
transmitting, by the one or more computing devices, the JSON file to the application on the client device in response to the query.

5. The method of claim 1, further comprising:
receiving, by the one or more computing devices and from the application on the client device, a request to update the FHIR resource, the non-FHIR resource, or the custom resource;
if the request is to update the FHIR resource, updating, by the one or more computing devices, the FHIR resource via the FHIR server;
if the request is to update the non-FHIR resource, updating, by the one or more computing devices, the non-FHIR resource via the non-FHIR server; and
if the request is to update the custom resource, updating, by the one or more computing devices, the FHIR resource of the custom resource via the FHIR server, and the non-FHIR resource of the custom resource via the non-FHIR server.

6. The method of claim 1, further comprising authenticating, by the one or more computing devices, the application or the client device prior to determining whether the query requests health data that is: the FHIR resource, the non-FHIR resource, or the custom resource.

7. The method of claim 1, further comprising authorizing, by the one or more computing devices, a user prior to determining whether the query requests health data that is: the FHIR resource, the non-FHIR resource, or the custom resource, to make sure that the authorized user has appropriate authorizations to access the data.

8. The method of claim 1, wherein the metadata table is dynamically configurable to define the relationship between the FHIR resource and the non-FHIR resource for the custom resource.

9. A non-transitory computer readable medium including instructions for a computing system for integrating health related data that when executed by the computing system, cause the computing system to perform the operations comprising:
receiving, by one or more computing devices and from an application on a client device, a query for health data;
determining, by the one or more computing devices, whether the query requests health data that is:
a Fast Healthcare Interoperability Resources (FHIR) resource,
a non-FHIR resource, or
a custom resource, wherein the custom resource is health data comprising a combination of the FHIR resource and the non-FHIR resource;
if determined that the query requests health data that is the FHIR resource, retrieving, by the one or more computing devices, the FHIR resource from a FHIR server via a GraphQL query or via a FHIR native REST API;
if determined that the query requests health data that is the non-FHIR resource, retrieving, by the one or more computing devices, the non-FHIR resource from a non-FHIR server;
if determined that the query requests health data that is the custom resource:
accessing, by the one or more computing devices, a metadata table, wherein the metadata table defines a relationship between the FHIR resource and the non-FHIR resource for the custom resource,
based on the defined relationship retrieving, by the one or more computing devices, the FHIR resource from the FHIR server using the GraphQL query or the FHIR native REST API, and the non-FHIR resource, and
combining the retrieved FHIR resource and the non-FHIR resource as the custom resource; and
transmitting, by the one or more computing devices, the retrieved FHIR resource, non-FHIR resource, or custom resource to the application on the client device in response to the query.

10. The non-transitory computer readable medium of claim 9, wherein the operations further comprise transmitting, by the one or more computing devices, the retrieved FHIR resource, non-FHIR resource, or custom resource to the client device in real-time from when the query is received.

11. The non-transitory computer readable medium of claim 9, wherein the computing system is implemented using a cloud computing service.

12. The non-transitory computer readable medium of claim 9, wherein the operations further comprise:
formatting, by the one or more computing devices, the retrieved FHIR resource, non-FHIR resource, or custom resource in a JSON file; and
transmitting, by the one or more computing devices, the JSON file to the application on the client device in response to the query.

13. The non-transitory computer readable medium of claim 9, wherein the operations further comprise:
receiving, by the one or more computing devices and from the application on the client device, a request to update the FHIR resource, the non-FHIR resource, or the custom resource;
if the request is to update the FHIR resource, updating, by the one or more computing devices, the FHIR resource via the FHIR server;
if the request is to update the non-FHIR resource, updating, by the one or more computing devices, the non-FHIR resource via the non-FHIR server; and
if the request is to update the custom resource, updating, by the one or more computing devices, the FHIR resource of the custom resource via the FHIR server, and the non-FHIR resource of the custom resource via the non-FHIR server.

14. The non-transitory computer readable medium of claim 9, wherein the operations further comprise authenticating, by the one or more computing devices, the application or the client device prior to determining whether the query requests health data that is: the FHIR resource, the non-FHIR resource, or the custom resource.

15. The non-transitory computer readable medium of claim 9, wherein the operations further comprise authorizing, by the one or more computing devices, a user prior to determining whether the query requests health data that is: the FHIR resource, the non-FHIR resource, or the custom resource, to make sure that the authorized user has appropriate authorizations to access the data.

16. The non-transitory computer readable medium of claim 9, wherein the metadata table is dynamically configurable to define the relationship between the FHIR resource and the non-FHIR resource for the custom resource.

17. A computing system for integrating health related data comprising:
a memory configured to store instructions;
a processor, coupled to the memory, configured to process the stored instructions to:
receive, from an application on a client device, a query for health data;
determine whether the query requests health data that is:
a Fast Healthcare Interoperability Resources (FHIR) resource,
a non-FHIR resource, or
a custom resource, wherein the custom resource is health data comprising a combination of the FHIR resource and the non-FHIR resource;
if determined that the query requests health data that is the FHIR resource, retrieve the FHIR resource from a FHIR server via a GraphQL query or via a FHIR native REST API;
if determined that the query requests health data that is the non-FHIR resource, retrieve the non-FHIR resource from a non-FHIR server;
if determined that the query requests health data that is the custom resource:
access a metadata table, wherein the metadata table defines a relationship between the FHIR resource and the non-FHIR resource for the custom resource,
based on the defined relationship retrieve the FHIR resource from the FHIR server using the GraphQL query or the FHIR native REST API, and the non-FHIR resource, and
combine the retrieved FHIR resource and the non-FHIR resource as the custom resource; and
a communications unit including microelectronics, coupled to the memory, configured to process the stored instructions to transmit the retrieved FHIR resource, non-FHIR resource, or custom resource to the application on the client device in response to the query.

18. The computing system of claim 17, wherein the communications unit is further configured transmit the retrieved FHIR resource, non-FHIR resource, or custom resource to the client device in real-time from when the query is received.

19. The computing system of claim 17, wherein the computing system is implemented using a cloud computing service.

20. The computing system of claim 17, wherein:
the processor is further configured to format the retrieved FHIR resource, non-FHIR resource, or custom resource in a JSON file; and
the communications unit is further configured to transmit the JSON file to the application on the client device in response to the query.

21. The computing system of claim 17, wherein the processor is further configured to:
receive, from the application on the client device, a request to update the FHIR resource, the non-FHIR resource, or the custom resource;
if the request is to update the FHIR resource, update the FHIR resource via the FHIR server;
if the request is to update the non-FHIR resource, update the non-FHIR resource via the non-FHIR server; and
if the request is to update the custom resource, update the FHIR resource of the custom resource via the FHIR server, and the non-FHIR resource of the custom resource via the non-FHIR server.

22. The computing system of claim 17, wherein the processor is further configured to authenticate the application or the client device prior to determining whether the query requests health data that is: the FHIR resource, the non-FHIR resource, or the custom resource.

23. The computing system of claim 17, wherein the processor is further configured to authorize a user prior to determining whether the query requests health data that is: the FHIR resource, the non-FHIR resource, or the custom resource, to make sure that the authorized user has appropriate authorizations to access the data.
